(19) United States
(12) Patent Application Publication
(10) Pub. No.: US 2013/0109995 A1
(43) Pub. Date: May 2, 2013

(54) METHOD OF BUILDING CLASSIFIERS FOR REAL-TIME CLASSIFICATION OF NEUROLOGICAL STATES

(71) Applicants: Neil S. Rothman, Baltimore, MD (US); Arnaud Jacquin, New York, NY (US); Leslie S. Prichep, Mamaroneck, NY (US); Samanwoy Ghosh Dastidar, Bethesda, MD (US); Julie Filipenko, New York, NY (US)

(72) Inventors: Neil S. Rothman, Baltimore, MD (US); Arnaud Jacquin, New York, NY (US); Leslie S. Prichep, Mamaroneck, NY (US); Samanwoy Ghosh Dastidar, Bethesda, MD (US); Julie Filipenko, New York, NY (US)

(21) Appl. No.: 13/284,184

(22) Filed: Oct. 28, 2011

(51) Int. Cl. A61B 5/0476

(52) U.S. Cl. 600/544

(57) ABSTRACT

A method of building binary classifiers for classification of brain electrical activity data into one or more neurological classes is described. The method comprises the steps of extracting quantitative features from the brain electrical activity data, and reducing the pool of extracted features into a computationally manageable and statistically relevant set of features which can then be used for designing one or more classifiers.

The present disclosure relates to the field of neurological assessment, and specifically, to the development of a method for building classifiers for classifying a patient into one or more neurological states based on the patient's acquired brain electrical signals.

All of the brain's activities, whether sensory, cognitive, emotional, autonomic, or motor function, is electrical in nature. The brain electrical activity establishes the basic signatures of the electroencephalogram (EEG) and creates identifiable frequencies which have a basis in anatomic structure and function. Understanding these basic rhythms and their significance makes it possible to characterize the electrical brain signals as being within or beyond normal limits. At this basic level, the electrical signals serve as a signature for both normal and abnormal brain function, and an abnormal brain wave pattern can be a strong indication of certain brain pathologies.

Currently, brain electrical activity data is collected and analyzed by an EEG technician, and is then presented to a neurologist for interpretation and clinical assessment. Manual review of EEG recordings for detection of abnormal electrographical patterns is time-consuming, subjective, and may be inaccurate. Further, the waveforms for many neurological conditions, such as, traumatic brain injury (TBI), cannot be seen directly on the EEG by the interpreting expert without additional signal processing. This makes the currently available EEG equipment inadequate for neuro-triage applications in emergency rooms or at other point-of-care settings. There is an immediate need for real-time objective evaluation of brain electrical signals in order to enable clinicians, EMTs or ER personnel, who are not well trained in neurodiagnostics, to easily interpret and draw diagnostic inferences from the data recorded at the point-of-care. This in turn will help the medical personnel in selecting an immediate course of action, prioritizing patients for imaging, or determining if immediate referral to a neurologist or neurosurgeon is required.

Objective assessment of brain electrical signals may be performed using a classifier that provides a mathematical function for mapping (or classifying) a vector of quantitative features extracted from the recorded data into one or more predefined categories. Classifiers are built by forming a training dataset, where each subject is assigned a “label,” namely a neurological class based on information provided by doctors and obtained with the help of state-of-the-art diagnostic systems, such as CT scan, MRI, etc. For each subject in the dataset, a large set of quantitative signal attributes or features (computed from the EEG) is also available. The process of building a classifier from a training dataset involves the selection of a subset of features (from the set of all quantitative features), along with the construction of a mathematical function which uses these features as input and which produces as its output an assignment of the subject's data to a specific class. After a classifier is built, it may be used to classify unlabeled data records as belonging to one or the other potential neurological classes. Classification accuracy is then reported using a testing dataset which may or may not overlap with the training set, but for which a priori classification data is also available. The accuracy of the classifier is dependent upon the selection of features that comprise part of the specification of the classifier. Well-chosen features may not only improve the classification accuracy, but also reduce the amount and quality of training data items needed to achieve a desired level of classification performance. However, the task of finding the “best” features may require an exhaustive search of all possible combinations of features, and computation and evaluation of each possible classifier. Therefore, most classification systems currently rely heavily on the art and experience of the (human) designer of the classifier for selecting the features that go into the classifier, which can be time-intensive, and can also result in subjectivity, or in missed solutions that may be better at classifying, and which can additionally be prone to human error.

The present disclosure addresses the need for a classification system for real-time evaluation of the brain electrical activity of a patient. A first aspect of the disclosure comprises a method of building classifiers to classify individuals into one of two neurological classes. The method comprises the steps of recording brain electrical signals from a plurality of individuals in the presence or absence of brain abnormalities using one or more neurological electrodes, extracting quantitative signal features from the recorded brain electrical signals, and storing the extracted signal features in a population reference database. The method further comprises the steps of applying one or more data reduction criteria to the stored features in the population reference database to create a reduced pool of signal features, selecting a subset of signal features from the reduced pool of features to construct the binary classifier, and then evaluating the performance of the binary classifier.

Another aspect of the present disclosure also includes a method of building binary classifiers to classify individual data into one of two categories. The method comprises the steps of providing a processor configured to build a binary classifier, accessing a pool of quantitative features from a population reference database stored in a memory device operatively coupled to the processor, applying one or more data reduction criteria to the pool of quantitative features to create a reduced pool of features that are statistically relevant to the classification, and selecting a subset of features from the reduced pool of features to construct the binary classifier.

It is to be understood that both the foregoing general description and the following detailed description are exemplary and explanatory only and are not restrictive of the invention, as claimed. The terms “EEG signal” and “brain electrical signal” are used interchangeably in this application to mean signals acquired from the brain using neurological electrodes.

The accompanying drawings, which are incorporated in and constitute a part of this specification, illustrate embodiments of the invention and together with the description, serve to explain the principles of the various aspects of the invention.

### BRIEF DESCRIPTION OF DRAWINGS

FIG. 1 is a flowchart of the classifier building process, in accordance with an exemplary embodiment of the present disclosure;

FIG. 2A is a ROC curve for an exemplary “4 vs. 3, 2, 1” classifier;

FIG. 2B is a histogram of discriminant scores for the exemplary “4 vs. 3, 2, 1” classifier referred to in FIG. 2A;

FIG. 3A is a ROC curve for an exemplary “1 vs. 2, 3, 4” classifier;

FIG. 3B is a histogram of discriminant scores for the exemplary “1 vs. 2, 3, 4” classifier referred to FIG. 3A;

FIG. 4A is a ROC curve for an exemplary “1, 2 vs. 3, 4” classifier; and

FIG. 4B is a histogram of discriminant scores for the exemplary “1, 2 vs. 3, 4” classifier referred to in FIG. 4A.

### DESCRIPTION OF ILLUSTRATIVE EMBODIMENTS

Reference will now be made in detail to certain embodiments consistent with the present disclosure, examples of which are illustrated in the accompanying drawings. Wherever possible, the same reference numbers will be used throughout the drawings to refer to the same or like parts.

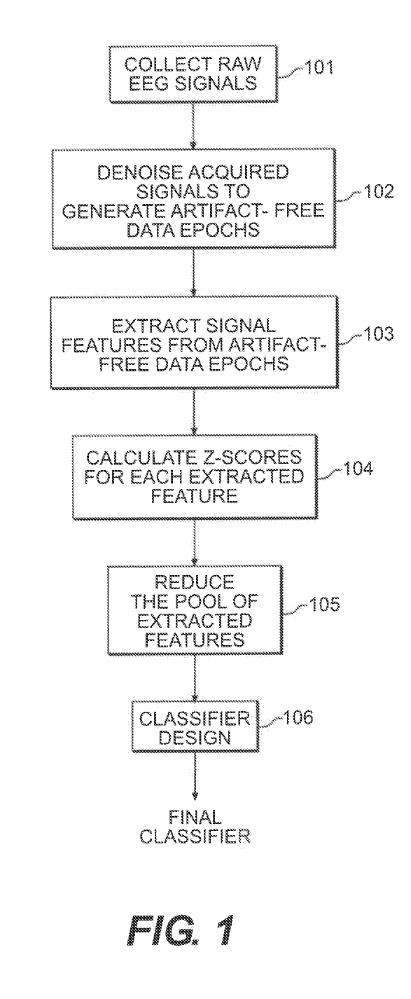

The present disclosure describes a method for building a binary classifier for mapping recorded brain electrical activity data into one or more predefined neurological classes or categories. An exemplary classifier building methodology is illustrated in FIG. 1. The classifier building algorithm, as illustrated in FIG. 1, is executed by a signal processing device comprising a processor. The first step in the classifier building process is collection of raw brain electrical signals (step 101). In an exemplary embodiment, a subject's electrical brain activity is recorded using a varying number of non-invasive neurological electrodes located at standardized positions on the scalp and forehead and ear-lobes. In one exemplary embodiment, a subject's brain electrical activity is recorded using an electrode array comprising at least one neurological electrode to be attached to a patient's head to acquire the brain electrical signals. The electrodes are configured for sensing both spontaneous brain activity as well as evoked potentials generated in response to applied stimuli (e.g. auditory, visual, tactile stimuli, etc.). In an exemplary embodiment, recording is done using five (active) channels and three reference channels. The electrode array consists of anterior (frontal) electrodes: Fp1, Fp2, F7, F8, AFz (also referred to as Fz′) and Fpz (reference electrode) to be attached to a subject's forehead, and electrodes A1 and A2 to be placed on the front or back side of the ear lobes, or on the mastoids, in accordance with the International 10/20 electrode placement system (with the exception of AFz). Other electrode configurations may be utilized as and when required, as would be understood by those of ordinary skill in the art.

In exemplary embodiments, the signal processor running the classifier building algorithm is configured to implement a artifact detection algorithm to identify data that is contaminated by non brain-generated artifacts, such as eye movements, electromyographic activity (EMG) produced by muscle tension, spike (impulse), external noise, etc., as well as unusual electrical activity of the brain not part of the estimation of stationary background state (step 102). By way of example, artifact identification is performed using as input the signals from the five active leads Fp1, Fp2, F7, F8, AFz referenced to linked ears (A1+A2)/2, and sampled at 100 Hz. In one exemplary embodiment, incoming EEG signals are split into sub-epochs of length 320 ms (32 data points per sub-epoch). Artifact identification is done on a per-sub-epoch basis and guard bands are implemented around identified artifact segments of each type. Artifact-free epochs are then constructed from continuous data segments, with each data segment being no shorter than 960 ms (which corresponds to the time span of 3 contiguous sub-epochs). In one embodiment, artifact-free or “denoised” data epochs having a temporal length of 2.56 seconds, which corresponds to 256 samples for data sampled at 100 Hz, are constructed by combining (for example, by an operation of concatenation, data overlapping, etc.) clean sub-epochs. The resulting artifact-free data epochs are then processed to extract quantitative signal features (step 103).

In an exemplary embodiment, the processor is configured to perform a linear feature extraction algorithm based on Fast Fourier Transform (FFT) and power spectral analysis, according to a method disclosed in commonly-assigned U.S. patent application Ser. Nos. 11/195,001 and 12/041,106, which are incorporated herein by reference in their entirety. In short, the algorithm computes quantitative features obtained using the Fast Fourier Transform (FFT), and calculates the spectral power at predefined frequency bands, along with other signal features. The frequency composition can be analyzed by dividing the signal into the traditional frequency bands: delta (1.5-3.5 Hz), theta (3.5-7.5 Hz), alpha (7.5-12.5 Hz), beta (12.5-25 Hz), and gamma (25-50 Hz). Higher frequencies, up to and beyond 1000 Hz may also be used. Univariate features are computed by calculating the absolute and relative power for each of the electrodes or between a pair of electrodes within selected frequency bands, and the asymmetry and coherence relationships among these spectral measurements within and between pairs of electrodes. The processor may also be configured to compute multivariate features, which are non-linear functions of groups of the univariate features involving two or more electrodes or pairs of electrodes or multiple frequency bands.

In another embodiment, the processor is configured to perform feature extraction based on wavelet transforms, such as Discrete Wavelet Transform (DWT) or Complex Wavelet Transforms (CWT). In yet another embodiment, the processor is configured to perform feature extraction using non-linear signal transform methods, such as wavelet packet transform, according to a method disclosed in commonly-assigned U.S. patent application Ser. No. 12/361,174, which is incorporated herein by reference in its entirety. The features extracted by this method are referred to as Local Discriminant Basis (LDB) features.

In another embodiment, diffusion geometric analysis is used to extract non-linear features according to a method disclosed in commonly-assigned U.S. patent application Ser. No. 12/105,439, which is incorporated herein by reference in its entirety. In yet another embodiment, entropy, fractal dimension and mutual information-based features are also calculated.

The computed measures per epoch are combined into a single measure of EEG signal per channel and transformed for Gaussianity. Once a Gaussian distribution has been demonstrated and age regression applied, statistical Z transformation is performed to produce Z-scores (step 104). The Z-transform is used to describe the deviations from age expected normal values:

$$Z = \frac{Subject Value - Norm for Age}{Standard Deviation for Age}$$

The Z-scores are calculated for each feature and for each electrode, pair of electrodes, or pair of a pair of electrodes, using a database of response signals from a large population of subjects believed to be normal, or to have other pre-diagnosed conditions. In particular, each extracted feature is converted to a Z-transformed score, which characterizes the probability that the extracted feature observed in the subject will conform to a normal value.

The age-regressed and Z-transformed signal features are stored in a population reference database. The database is stored in a memory device that is operationally coupled to the signal processor executing the classifier building algorithm. In one embodiment, the population reference database comprises population normative data indicative of brain electrical activity of a first plurality of individuals having normal brain state, or population reference data indicative of brain electrical activity of a second plurality of individuals having an abnormal brain state. In another embodiment, the database comprises features from the subject's own brain electrical activity data generated in the absence or presence of an abnormal brain state. The population reference database employed by the inventor has been shown to be independent of racial background and to have extremely high test-retest reliability, specificity (low false positive rate) and sensitivity (low false negative rate). The weights and constants that define a classification function (such as, Linear Discriminant Function, Quadratic Discriminant Function, etc.) are derived from a set of quantitative signal features in the population reference database. Thus, the design or construction of a classification function targeting any classification task (e.g. “Normal” vs. “Abnormal” brain function) requires selection of a set of features from a large available pool of features in the population reference database. The selection of the “best” features results in the “best” classification performance, characterized by, for example, the highest sensitivity/specificity and lowest classification error rates. In order to make the feature selection process more efficient and to ensure higher classification performance, the available pool of features from the population reference database must be transformed or reduced to a computationally manageable and neurophysiologically relevant pool of features from which a subset of features for a particular classification task may be selected during classifier construction.

Accordingly, the next step in the classifier builder algorithm is reducing the pool of available features in the population reference database into a smaller set of features that contribute directly to a specific classification task (step 105). In an exemplary embodiment, a reduced pool of features is created using an “informed data reduction” technique, which relies on the specific downstream application of the classifier, neurophysiology principles and heuristic rules. In exemplary embodiments, the “informed data reduction” method includes several different criteria to facilitate the inclusion of features that most effectively provide separation among the classes. For example, in some embodiments, a data quality review is performed on the recorded EEG measures. If visual inspection reveals excessive noise or atypical data in any EEG measure, the features extracted from those EEG measures are excluded. In other embodiments, outliers are identified using the z-scores of the features. For example, in one embodiment, features with z-scores that are 6 standard deviations away from the mean value in a “normal” patient distribution are identified as outliers and excluded. Similarly, in another embodiment, features with z-scores that are 8 standard deviations away from the mean value in an “abnormal” patient distribution are excluded.

In certain embodiments, the “informed data reduction” method requires that each feature be replicable, i.e., it should provide approximately the same value in different temporal segments of the same recording, or across successive measurements of brain electrical signals performed on the same person's head. This ensures stability of the feature for multiple recordings. In one exemplary embodiment, feature replicability is quantified using a subset of data from the population reference database for which the features values are computed twice, during a first time period t₁ and during a second time period t₂, immediately following t₁. The replicability of any feature is derived from the mean value of the magnitude of the difference between the two instances of this feature during time periods t₁ and t₂. The features with low replicability values are excluded during the data reduction process.

In illustrative embodiments, a specific set of features is excluded during the data reduction process. For example, in some embodiments, all features in the Delta1 band are excluded due to the unreliability and lack of resolution of features computed in this frequency band. In other embodiments, all mean frequency features in the Beta2 band and Gamma band are excluded. In some other embodiments, all features in the Gamma band, except for phase and coherence variables, are excluded.

In another exemplary embodiment, the informed data reduction method invokes a criterion which requires separability of the feature distribution across the two groups for each binary classifier. In some embodiments, the Kolmogorov-Smirnov (KS) test is applied to test for separability. The features that fail the KS test are excluded to ensure that the distributions of each variable for the “more normal” category (of the two categories in the classifier) are significantly different from those of the “less normal” category. In the context of the present disclosure, a “more normal” category refers to the classification category that represents a population group having brain electrical activity that is functionally closer to the population normative data. For example, in a binary classifier designed to separate the class formed by combining the normal patients and patients with less severe functional brain injury (“brain state A”) from the class formed by combining patients with more severe functional injury and patients with structural injury (“brain state B”), the “brain state A” category is referred to as the “more normal” category.

In yet another exemplary embodiment, the informed data reduction method ensures that the mean value of any feature for the “more normal” population lies closer to the mean value for the normative population (i.e. mean=0, standard deviation=1) than do the mean values of any feature in the “less normal” population. For example, in a “normal” vs. “abnormal” brain function classification, this criterion ensures that the absolute mean value of a feature in the “normal” population is less than the absolute mean value of the feature in the “abnormal” population. Further, in some embodiments, a maximum value is set for the difference between the absolute mean value of a feature in the “more normal” group from the normative mean value (i.e. 0). In exemplary embodiments, this maximum value is set at 1.0, and a feature in the “more normal” category is excluded from the selection process if the absolute mean value is greater than 1.

In further exemplary embodiments, the informed data reduction method ensures the statistical separability of the feature distribution across subject categories by truncating the distributions of each quantitative feature to minimize the influence of outliers. In one illustrative embodiment, feature distribution is clipped at ±3.29 sigma (standard deviation) to ensure that the process of feature selection for each discriminant function is not overwhelmed by the presence of outliers.

Referring again to FIG. 1, once all the data reduction criteria are applied, the remaining reduced pool of features is utilized to design a binary classifier (step 106). In exemplary embodiments, a binary classifier is designed by selecting a specific set of features for each discriminant function based on performance. The search for the “best” features for a binary classification task is performed using a fully-automated system (hereinafter “classifier builder”), implemented as a computer program, the output of which is a Discriminant Function classifier. In exemplary embodiments, identification of the “best” features for a particular classification task is performed by computing multiple classifiers using different combination of features, and evaluating each possible classifier using an “objective function” that is directly related to classification performance. In exemplary embodiments, the classifier performance is tested using an objective function that is directly related to classification performance. In an exemplary embodiment, the objective function (figure of merit) used by a feature selection algorithm is the area under the Receiver Operating Characteristics (ROC) curve of a Discriminant Function, which is usually referred to as “Area Under the Curve” (AUC). For a given discriminant-based binary classifier, the ROC curve indicates the sensitivity and specificity that can be expected from the classifier at different values of the classification threshold T. Once a critical value (or threshold) T is selected, the output of the test becomes binary, and sensitivity and specificity for that particular threshold can be calculated. The ROC is the curve through the set of points: {(1-specificity(T), sensitivity(T))}, which is obtained by varying the value of the threshold T in fixed increments between 0 and 100. After the ROC curve is obtained, the area under the ROC curve (AUC) is calculated. AUC is a single number between 0 and 1, which reflects, jointly, the sensitivity and specificity of a binary classifier. Thus, AUC provides a quantitative global measure of achievable classifier performance.

In one exemplary embodiment, the search for the “best” features for a binary classification task is performed using a feature selection algorithm that is referred to herein as “Simple Feature Picker” (SFP) algorithm. The SFP algorithm selects a first feature by evaluating all features in the database, and selecting the feature that provides the best classifier performance. Subsequent features are selected to give the best incremental improvement in classifier performance.

In another exemplary embodiment, the SFP algorithm adds multiple features to the classifier at each iteration, calculates AUC of the resulting classifier at each iteration step, and selects the features that provide that greatest improvement in AUC.

In yet another exemplary embodiment, feature selection is performed using one or more evolutionary algorithms, for example, a Genetic Algorithm (GA), as described in commonly-owned U.S. application Ser. No. 12/541,272 which is incorporated herein by reference in its entirety. In another exemplary embodiment, the search for candidate features is performed using an optimization method, for example, Random Mutation Hill-Climbing (RMHC) method, or Modified Random Mutation Hill Climbing (mRMHC), which can be used in a stand-alone fashion or can be combined with the GA algorithm or SFP algorithm (for example, as a final “local search” to replace one feature by another to improve the final feature subset), as further described in the U.S. application Ser. No. 12/541,272 incorporated herein.

The classifier design process (step 106, FIG. 1) also includes the selection of the type of discriminant function that would provide the best performance for a specific binary classification task. In exemplary embodiments, the classification function is a Linear Discriminant Function (LDF), which provides optimum classification results for subject categories that have clear differences in mean values of the features. In exemplary embodiments, a Linear Discriminant Function optimally combines the features (Z-scores) into a discriminant output/score that possesses the maximum discriminating power. In one embodiment, the discriminant function of a binary classifier assigns for each given subject a discriminant score (a real-valued number) between 0 and 100. The classification rule which is commonly associated with Linear Discriminant Functions is the following: after a cut-off threshold T is selected (for example, but not necessarily, in the middle of the discriminate score range i.e. T=50), the classifier assigns any subject with a discriminant score g≦T to the category “brain state A” and assigns any subject with a score g>T to the category “brain state B.” A score “lower than or equal to 50” indicates that the subject is more likely to belong to brain state A than to brain state B, and vice versa. Examples of different classification classes include, but are not limited to, “normal brain function” vs. “abnormal brain function”, “organic brain dysfunction” vs. “functional brain dysfunction”, “focal brain dysfunction” vs. “diffuse brain dysfunction”, “normal brain function” vs. “(closed-head) traumatic brain injury (TBI),” “normal brain function” vs. “mild TBI (concussion)”, etc.

In other exemplary embodiments, non-linear discriminant functions are built from a training dataset through selection of a subset of features (from the reduced set of quantitative features). Examples of non-linear classification functions include Quadratic Discriminant Functions (QDF). QDFs are particularly efficient for classification tasks where the subject categories overlap and/or have differences in both mean and standard deviation of feature values.

Depending on the type of discriminant function, the classifier builder puts a limit on the maximum number of features to be used for classifier construction in order to ensure classifier performance for a broader population group outside the training dataset. For example, in the construction of linear discriminant functions, the number of features used is less than one tenth of the number of subjects in the overall training group. In quadratic discriminant functions, the number of features (n) used in classifier construction is selected such that n(n+3)/4 is less than the smallest group on either side of the classifier.

In certain embodiments, a series of binary classifiers that use either linear or non-linear discriminant functions are used to classify individuals into multiple categories. In some embodiments, x-1 discriminant functions are used to separate individual subjects into x classification categories. In an exemplary embodiment, three binary classifiers are designed and implemented for classifying patients into one of four categories related to the extent of brain dysfunction resulting from a traumatic brain injury (TBI), as described in U.S. application Ser. No. 12/857,504, which is incorporated herein by reference.

In alternative embodiments, a single binary classifier is used to perform a three-way classification task by executing the classifier twice in parallel or in cascade. The binary classifier may use either a linear or non-linear discriminant function designed by selecting a feature subset from the training dataset. For the construction of a binary classifier in a three-way classification task, two different values for the cut-off threshold T are selected to indicate different levels of sensitivity and specificity that can be expected from a classifier for the two separate classification tasks (i.e., the classification of “brain state A” from “brain state B,” and the classification of “brain state B” from “brain state C”). The feature subset for the final classifier is selected based on the classification performance for all three categories.

After a classifier is built, classification accuracy is evaluated using a testing dataset for which gold standard classification data is available. In some embodiments, the testing dataset is separate from the training set. In some other exemplary embodiments, all available data is used for both training and testing of the classifier. In such embodiments, performance of the classifier is evaluated using 10-fold and/or leave-one-out (LOO) cross-validation methods. In exemplary embodiments, two separate cross-validation method are applied for feature selection and determining the overall performance of the classifier with the selected subset of features. In illustrative embodiments, the 10-fold cross-validation method is used for feature selection and the LOO cross-validation method is applied for testing the overall performance. The subset of features found using the 10-fold method is applied to the remaining subjects in the testing database and a decision threshold that provides a target level of performance with respect to sensitivity (true positive rate) is selected. The decision threshold is selected as the discriminant function value that separates the two classification categories in the binary classifier with a sensitivity equal to the target sensitivity. The process is repeated for all subjects in the database and the sensitivity and specificity of classification is calculated for each subject.

In exemplary embodiments, the classifier builder utilizes additional localized optimization methods to refine the final subset of features in each classifier. For example, in some embodiment, the selection of a particular subset of features is performed using “Partial Area Under the Curve” (partial AUC) as an objective function (figure of merit), which includes only a specific portion of the ROC curve of a Discriminant Function. In illustrative embodiment, optimization is focused only in the region of the ROC curve that includes the target sensitivity and specificity values. The additional optimization methods are applied either as a part of the feature selection process, or after the completion of the cross-validation tests. After a classifier is built and tested for accuracy, it may be used to classify unlabeled data records as belonging to a particular diagnostic class.

### Example: Application of Three Binary Classifiers for Differential Classification of Extent of Brain Dysfunction

In an exemplary embodiment of the present disclosure, three Quadratic Discriminant Functions (QDF) are designed and implemented for classifying patients into one of four categories related to the extent of brain dysfunction resulting from a traumatic brain injury (TBI). As would be understood by a person of ordinary skill in the art, any other type of linear or non-linear classifier (for example, Linear Discriminant Analysis, Gaussian Mixture Model, etc.) could also be used to classify the patients if clinically acceptable classification performance could be achieved. The four categories relating to the presence and severity of TBI are described in commonly-owned U.S. application Ser. No. 12/857,504, which is incorporated herein by reference in its entirety. In short, category 1 relates to normal brain activity, category 2 relates to mild TBI, category 3 relates to moderate TBI, and category 4 relates to structural brain injury requiring immediate treatment. The three quadratic classifiers designed to classify a patient into one of the four categories are defined as follows: classifier 1 (referred to herein as “1 vs. 2,3,4”) is intended to separate the class of normal patients from the class of abnormal patients; classifier 2 (referred to herein as “1,2 vs. 3,4”) is intended to separate the class formed by combining the normal patients and patients with less severe functional brain injury from the class formed by combining patients with more severe functional injury and CT+ patients (patients with structural injury); and, classifier 3 (referred to herein as “4 vs. 3,2,1”) is intended to separate the class formed by all patients who are or are expected to be CT− (patients without structural injury) from the class of CT+ patients.

A processor running the classification algorithm is configured to execute the three classifiers independently of each other, and provide three separate classification results along with some objective performance measures for each classifier. The classification decision is then driven by a clinician based on the classification performance and other clinically relevant factors, such as, symptoms presented, history of injury, etc. The performance of the three classifiers were tested by computing the specificity (true negative rate) and sensitivity (true positive rate) and the correct classification rates in each of the four categories. ROC curves were used to illustrate quantitatively the performance of each binary classifier, and to compute the specificity and sensitivity values. This allows, for example, a threshold T to be selected that ensures that a conservative classification is always assigned according to the appropriate stratification of risk for the categories being separated.

The training dataset used to design the three classifiers comprised a total of 688 subjects. The breakdown of subjects in each of the four categories related to the extent of brain dysfunction was as follows:

| Category | No. of subjects |
|---|---|
| 4 | 109 |
| 3 | 143 |
| 2 | 157 |
| 1 | 279 |

The maximum number of features in each QDF was calculated using the formula n(n+3)/4<M, where n is the number of features allowed and M is the number of subjects in the smallest group on either side of the discriminant. Based on this formula, the maximum number of features for each discriminant function was as follows:

| | Maximum no. | |
|---|---|---|
| Classifier | of features | Justification |
| 1 vs. 2, 3, 4 | 31 | 31*34/4 < 279 |
| 1, 2 vs. 3, 4 | 30 | 30*34/4 < 252 |
| 4 vs. 3, 2, 1 | 19 | 9*22/4 < 109 |

All features were z-transformed relative to age expected normal values and the available pool of features in the training dataset was then reduced to a statistically relevant set of features using the “informed data reduction” method describe in the present disclosure. The quadratic discriminant functions were then designed using a combination of the “Simple Feature Picker” (SFP) algorithm, genetic algorithm and Random Mutation Hill Climbing algorithm. Classification performance was expressed in terms of sensitivity and specificity using area under the ROC curve (AUC) as an objective function.

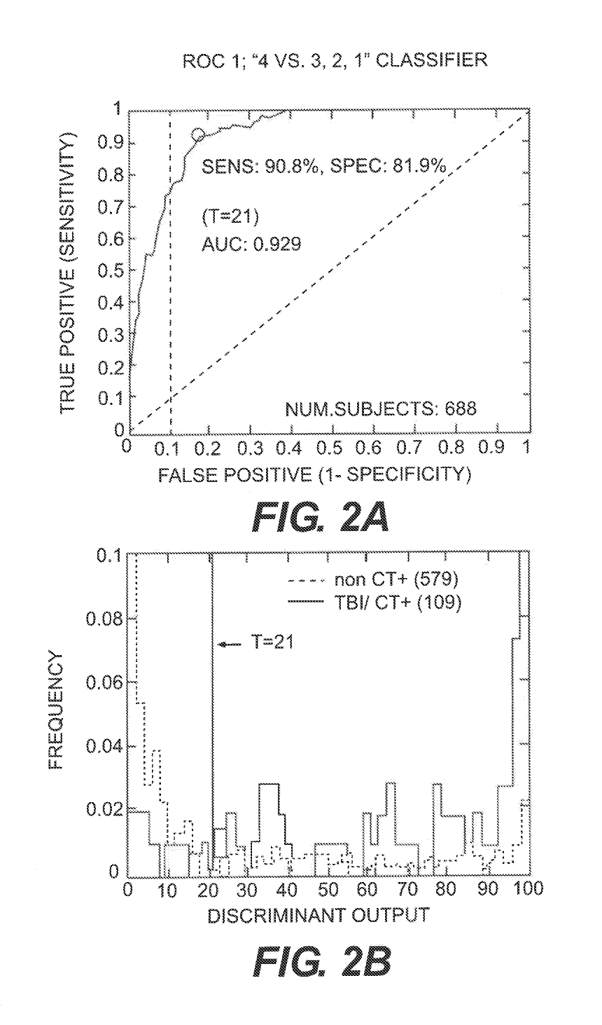

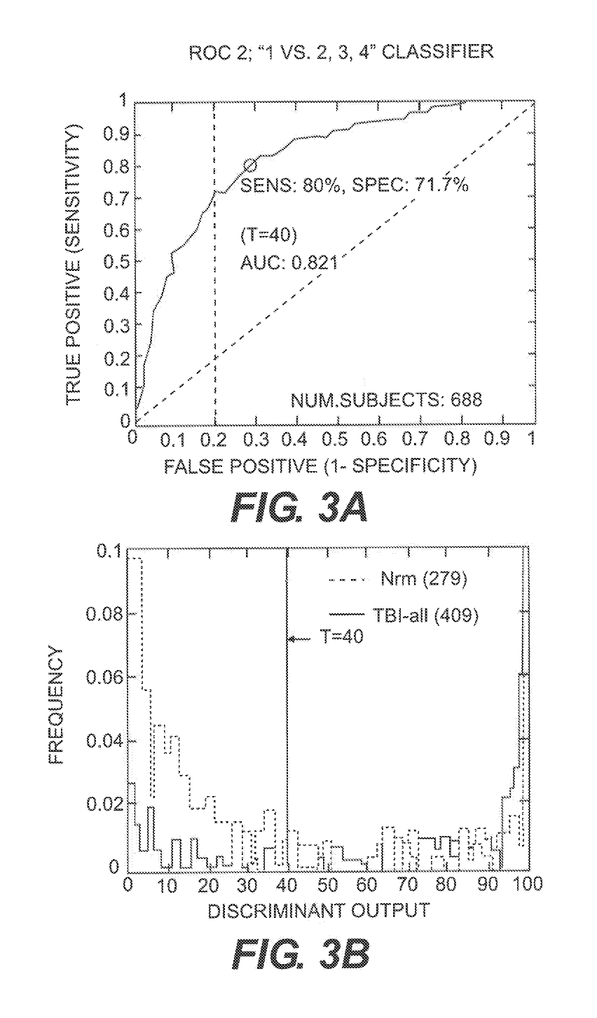

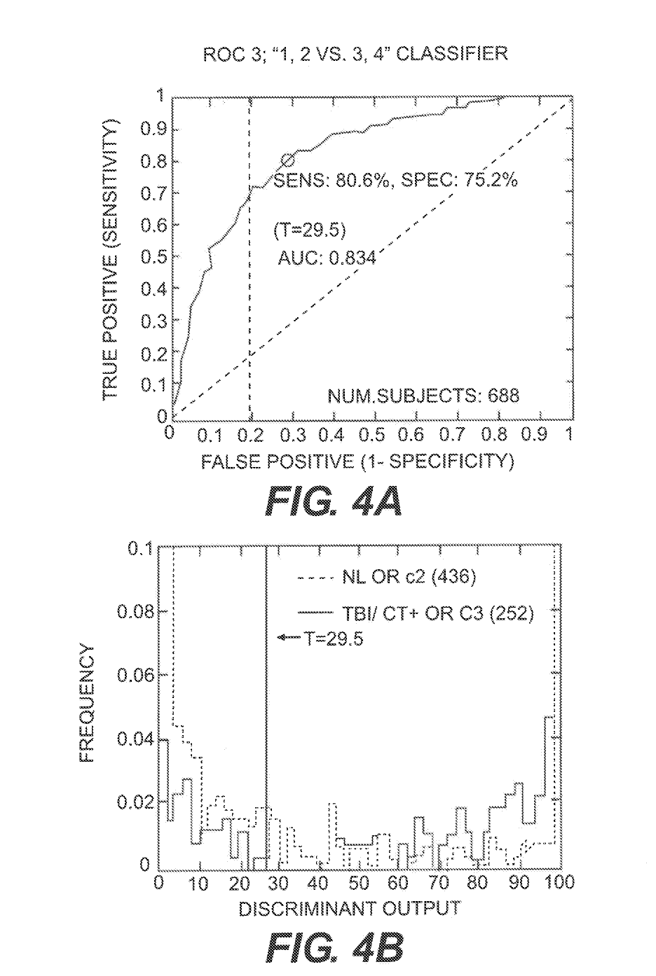

FIGS. 2A and 2B illustrate a ROC curve and histogram of discriminant scores for a “4 vs. 3, 2, 1” classifier. The ROC curve demonstrates the achievable statistical performance of the classifier for a threshold value T=21. The threshold T=21 was selected to achieve the highest sensitivity and specificity for the classification calculated using the LOO method. After the ROC curve is obtained, the area under the ROC curve (AUC) is calculated, which represents the surface area of the region located under the ROC curve and jointly reflects the sensitivity and specificity of a binary classifier. Category 4 was separated from all other categories with a sensitivity of 90.8% and a specificity of 80.9% (AUC=0.929) Similarly, FIGS. 3A and 3B illustrate ROC curves and histograms of discriminant scores for a “1 vs. 2, 3, 4” classifier and a “1, 2 vs. 3, 4” classifier, respectively. As shown in the figures, category 1 was separated from all others with a sensitivity of 80% and a specificity of 71.7% (AUC=0.821), and category 3 and 4 (those needing further observation or immediate triage) were separated from categories 2 and 1 (those who could be considered to be returned to activity, with or without recommendation for follow-up) with a sensitivity of 80.6% and specificity of 75.2% (AUC=0.834). In sum, the quadratic classifiers designed using the data reduction method described in this disclosure demonstrated high sensitivity and specificity in identification of TBI requiring immediate triage, as well as in the separation of those with head injuries that have different levels of brain dysfunction.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of building a binary classifier for classifying subjects into one of two brain function categories, comprising the steps of:
providing a signal processing device operatively connected to a memory device storing a population reference database, the signal processing device comprising a processor configured to perform the steps of:
obtaining brain electrical signals in machine readable format from the population reference database, wherein the signals are recorded from a plurality of individuals in the presence or absence of brain abnormalities using one or more neurological electrodes;
extracting quantitative signal features from the recorded brain electrical signals;
storing the extracted signal features in the population reference database;
applying one or more data reduction criteria to the stored features in the population reference database to create a reduced pool of signal features;
selecting a subset of signal features from the reduced pool of features to construct the binary classifier; and
determining classification accuracy of the binary classifier by using it to classify data records having a priori classification information.

2. The method of claim 1, wherein the one or more data reduction criteria comprises a measure of the replicability of the features.

3. The method of claim 1, wherein the one or more data reduction criteria includes identification of outliers using z-scores of the features.

4. The method of claim 1, wherein the one or more data reduction criteria comprises a measure of the separability of the features across the two brain function categories.

5. The method of claim 1, wherein the one or more data reduction criteria includes exclusion of a specific class of features.

6. The method of claim 5, wherein all features in the Delta1 band are excluded.

7. The method of claim 5, wherein all mean frequency features in the Beta2 band and Gamma band are excluded.

8. The method of claim 1, wherein the subset of features are selected using an evolutionary algorithm.

9. The method of claim 8, wherein the evolutionary algorithm applied is a genetic algorithm.

10. The method of claim 8, wherein the selected subset of features is optimized using at least one of a Random Mutation Hill Climbing algorithm and a Modified Random Mutation Hill Climbing algorithm,

11. The method of claim 1, wherein the subset of features is selected using a Simple Feature Picker algorithm

12. The method of claim 11, wherein the selected subset of features is optimized using at least one of a Random Mutation Hill Climbing algorithm and a Modified Random Mutation Hill Climbing algorithm.

13. The method of claim 1, wherein the binary classifier is a Linear Discriminant Function.

14. The method of claim 1, wherein the binary classifier is a Quadratic Discriminant Function.

15. The method of claim 1, wherein the quantitative signal features are derived from the brain electrical signals using wavelet transformation.

16. The method of claim 1, wherein the quantitative signal features are derived from the brain electrical signals using Fast Fourier Transformation.

17. The method of claim 1, wherein an objective function is used to evaluate the performance of the binary classifier.

18. The method of claim 17, wherein the objective function used is Area Under the Receiver Operating Curve of the binary classifier.

19. The method of claim 17, wherein the objective function used is Partial Area Under the Receiver Operating Curve of the binary classifier.

20. A method of building a binary classifier for classification of individual data into one of two categories, comprising the steps of:
providing a processor configured to build a binary classifier;
accessing a pool of quantitative features from a population reference database stored in a memory device operatively coupled to the processor;
applying one or more data reduction criteria to the pool of quantitative features;
creating a reduced pool of features that are statistically relevant to the classification;
selecting a subset of features from the reduced pool of features to construct the binary classifier; and
evaluating performance of the binary classifier using pre--labeled data records stored in the memory device, wherein the pre-labeled data records are assigned a priori to one of the two categories.

21. The method of claim 20, wherein the population reference database comprises brain electrical activity data from a plurality of individuals in the presence or absence of brain abnormalities.

22. The method of claim 21, wherein the brain electrical activity data is collected using an electrode array comprising at least one neurological electrode.

23. The method of claim 21, wherein the processor is configured to perform automatic identification and removal of artifacts from the brain electrical activity data.

24. The method of claim 20, wherein the one or more data reduction criteria comprises heuristic rules.

25. The method of claim 20, wherein the one or more data reduction criteria is based on neurophysiological principles.

26. The method of claim 20, wherein the one or more data reduction criteria comprises a measure of the replicability of the features.

27. The method of claim 20, wherein the one or more data reduction criteria includes identification of outliers using z-scores of the features.

28. The method of claim 20, wherein the one or more data reduction criteria comprises a measure of the separability of the features across the two categories.

29. The method of claim 20, wherein the one or more data reduction criteria includes exclusion of a specific class of features.

30. The method of claim 20, wherein a series of binary classifiers are used to classify the individual data into more than two categories.

31. The method of claim 30, wherein n-1 binary classifiers are used to classify the individual data into n categories.

32. The method of claim 31, wherein three binary classifiers are used to classify the individual data into four categories related to the extent of brain dysfunction following a traumatic brain injury.

33. The method of claim 20, wherein a single binary classifier is used to classify the individual data into more than two categories.

34. The method of claim 33, wherein the features are selected based on the classification performance of the binary classifier for all the categories.
